(19) United States
(12) Patent Application Publication
(10) Pub. No.: US 2010/0256446 A1
(43) Pub. Date: Oct. 7, 2010

(54) MEDICAL SCOPE CARRIER AND SCOPE AS SYSTEM AND METHOD

(75) Inventor: Gottumukkala S. Raju, League City, TX (US)

Correspondence Address: LOCKE LORD BISSELL & LIDDELL LLP, 600 TRAVIS SUITE 2800, HOUSTON, TX, 77002-3095, US

(73) Assignee: BOARD OF REGENTS, THE UNIVERSITY OF TEXAS SYSTEM, Austin, TX (US)

(21) Appl. No.: 12/599,658

(22) PCT Filed: May 9, 2008
(86) PCT No.: PCT/US08/63272

§ 371 Date: Nov. 10, 2009

Related U.S. Application Data
(60) Provisional application No. 60/917,437, filed on May 11, 2007.

(51) Int. Cl. A61B 1/01

(52) U.S. Cl. 600/114

(57) ABSTRACT

The present disclosure provides a scope carrier that contains a channel for a scope to be inserted therethrough. The scope can be coupled to the scope carrier so that during insertion of the carrier, the objective lens of the scope is used to guide the scope carrier. When the carrier is properly positioned, the scope can be released from the carrier and continue further through the body passages to the intended tissue or structure. The scope carrier can have a relatively much larger channel as a percentage of the carrier outer diameter than a typical scope. The carrier channel can be at least 50% of the scope carrier outer diameter. The carrier channel allows use of a larger scope to be inserted therethrough than has typically been available. The corresponding larger channel of the larger scope allows instruments to be inserted therethrough that heretofore have been unavailable in such combinations.

### CROSS REFERENCE TO RELATED APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO APPENDIX

Not applicable.

### BACKGROUND

1. Field of the Invention

The present disclosure relates to medical devices and procedures. More particularly, the disclosure relates to endoscopes used for medical procedures that can extend in length.

2. Description of Related Art

During current medical procedures, it is often advantageous to insert a scope into a patient's body through a body passage. For example, a scope can be inserted through an esophagus, through the stomach, and into the duodenum and to the subsequent jejunum of the small intestine. The scope can be used to view the condition of the linings or other tissue, and can be used to transport instruments through a longitudinal channel in the scope for performing various medical procedures, such as resection, excision, grasping, suturing, clamping, and other procedures. The outer scope diameter is limited by physiological considerations. With the addition of a light guide, objective lens, water channel, suction tube, and other customary structures associated with the scope, the channel of the scope through which the tools are transported is relatively small, on the order of a one to a few millimeters (mm).

If the scope is to be positioned into body passages that are curved or small, the larger sized scopes may be unsuitable to travel into or through such body passages. The rigidity of such larger scopes may have difficulty bending into the curved passages and can cause risk of perforation of the tissue forming the passageway. Some scopes are able to bend at the tip through manipulation of guide wires external to the body passageway by the physician performing the procedure. While such capabilities extend the scope, the procedure can at times lack sufficient maneuverability to travel the full needed distance and the intended procedure fails. The patient is sometimes relegated to another, later procedure such as through a different passageway.

Using a smaller, more flexible scope in the first instance does not often provide an acceptable alternative. The flexibility of the smaller scope, while suitable for bending through specific curved portions of the body passages, may be too flexible over the entire distance. Such flexibility can lead to looping in larger passages, such as the stomach, and therefore can be unsuitable.

Some efforts have been made with a “mother-daughter” scope assembly, also referred to as a “baby scope.” In this assembly, the larger “mother” scope with its full assortment of optics, channels and guides, is pushed through the body passageway to a certain point and then a smaller “daughter” scope with its optics and channels is pushed through the channel of the mother scope into smaller passages. This arrangement provides additional length to the procedure, but the available size of the mother channel places a practical limit on the outer diameter of the daughter scope. Thus, very small daughter scopes are generally used that in turn have even smaller channels that restrict the type of tools that can travel down the daughter channel. Often, the daughter scopes are relegated to visual observations with its optics rather than resection, grasping, suturing, clamping, and other procedures that are also needed and generally limit the usefulness of the daughter scope.

To overcome some of the mother-daughter limitations, a system is commercially available that inserts a scope partially into the body passage and slides a sheath over the scope already in the body passage to stiffen the scope for potential further penetration into the passage. The sheath is self guided along the outer diameter of the scope and depends on the scope being navigated in the body passage to some desired point so that then the sheath can be slid down the scope.

Thus, there remains a need for an improved scope insertable through body passages that can provide both rigidity and flexibility.

### BRIEF SUMMARY

In this field, special and sometimes simple devices from the viewpoint of hindsight can yield major improvements in costs, time, or the ability to even perform a desired medical procedure. The present disclosure provides an improved method and device for better inserting an endoscope into a body passage.

The present disclosure provides a scope carrier that contains a channel for a scope to be inserted therethrough. The combination of the scope carrier and scope allows a stiffer outer scope carrier to be inserted a distance into a body passage and then a more flexible scope to continue into the body passage a greater distance. The scope can be coupled to the scope carrier so that during insertion of the carrier, the objective lens of the scope is used to guide the scope carrier. When the carrier is properly positioned, the scope can be released from the carrier and continue further through the body passages to the intended tissue or structure. The scope carrier can have a relatively much larger channel as a percentage of the carrier outer diameter than a typical scope. The carrier channel can be at least 50% of the scope carrier outer diameter. The carrier channel allows use of a larger scope to be inserted therethrough than has typically been available. The corresponding larger channel of the larger scope allows instruments to be inserted therethrough that heretofore have been unavailable in such combinations. The larger scope channel can be used to not only insert instruments therethrough, but also to retrieve material from the inserted scope, including biopsies, resected tissue, and so forth. The system can be used for examining small bowels or examining a colon that cannot be reached with conventional scopes. Further, the system can be used to examine the bile ducts or pancreas ducts, right colon or various convoluted angles of the stomach or colon that heretofore are complicated due to multiple bending functions.

The disclosure provides a medical endoscope system, comprising: a scope carrier comprising a cylindrical tube having a carrier channel formed therein and adapted to allow a scope to be inserted therethrough; and a scope comprising an objective lens and a scope channel, the scope being adapted to be slidably disposed within the carrier channel.

The disclosure provides a medical endoscope system, comprising a scope carrier comprising a cylindrical tube having a carrier channel formed therein and adapted to allow an scope to be inserted therethrough, the carrier channel being at least 70% of the cross sectional square area of an outer diameter of the scope carrier.

The disclosure further provides a method of inserting a medical scope and scope carrier into a body passage, comprising: inserting a medical scope into a scope carrier; guiding the scope carrier into the body passage using an objective lens on the scope to guide the scope carrier; positioning the scope carrier; and extending the scope from the scope carrier to travel further into the body passage.

### BRIEF DESCRIPTION OF THE DRAWINGS

While the concepts provided herein are susceptible to various modifications and alternative forms, only a few specific embodiments have been shown by way of example in the drawings and are described in detail below. The figures and detailed descriptions of these specific embodiments are not intended to limit the breadth or scope of the concepts or the appended claims in any manner. Rather, the figures and detailed written descriptions are provided to illustrate the concepts to a person of ordinary skill in the art as required by 35 U.S.C. §112.

FIG. 1 is a schematic diagram of an exemplary endoscope.

FIG. 2 is a schematic diagram of an exemplary longitudinal cross-section of a distal end of the endoscope of FIG. 1.

FIG. 2A is a schematic diagram of a distal end of the endoscope of FIG. 1.

FIG. 3 is a schematic perspective diagram of an exemplary endoscopic system of the present invention with a scope carrier and a scope inserted therein.

FIG. 4 is a schematic longitudinal, cross-sectional diagram of an exemplary embodiment of the scope carrier having a distal tip with an elevator.

FIG. 5 is a schematic longitudinal, cross-sectional diagram of the exemplary scope carrier having angulation capabilities.

FIG. 6 is a schematic longitudinal, cross-sectional diagram of an angulation structure of the scope carrier of FIG. 5.

FIG. 7 is a schematic diagram of supply sources for the scope.

FIG. 8 is a schematic diagram of the exemplary scope carrier and scope.

FIG. 9 is a schematic longitudinal, cross-sectional diagram of an exemplary endoscopic system.

FIG. 10 is a schematic longitudinal, cross-sectional diagram of the endoscopic system with an elevator and angulation controls.

FIG. 11 is a schematic longitudinal, cross-sectional diagram of the scope carrier having a light-image capture element.

FIG. 11A is a schematic longitudinal, cross-sectional diagram of the scope carrier showing an additional light-image capture element from FIG. 11.

FIG. 12 is a schematic cross-sectional diagram of an exemplary system having the scope carrier and the scope.

FIG. 13 is a schematic cross-sectional diagram of the exemplary system with an elevator.

FIG. 14 is a schematic cross-sectional diagram of the exemplary system with a plurality of elevators.

FIG. 15 is a schematic cross-sectional view of the endoscopic system with rollers.

FIG. 16 is a schematic cross-sectional view of the endoscopic system with inflatable portions.

### DETAILED DESCRIPTION

One or more illustrative embodiments of the concepts disclosed herein are presented below. Not all features of an actual implementation are described or shown in this application for the sake of clarity. It is understood that in the development of an actual embodiment, numerous implementation-specific decisions must be made to achieve the developer's goals, such as compliance with system-related, business-related and other constraints, which vary by implementation and from time to time. While a developer's efforts might be complex and time-consuming, such efforts would be, nevertheless, a routine undertaking for those of ordinary skill in the art having benefit of this disclosure.

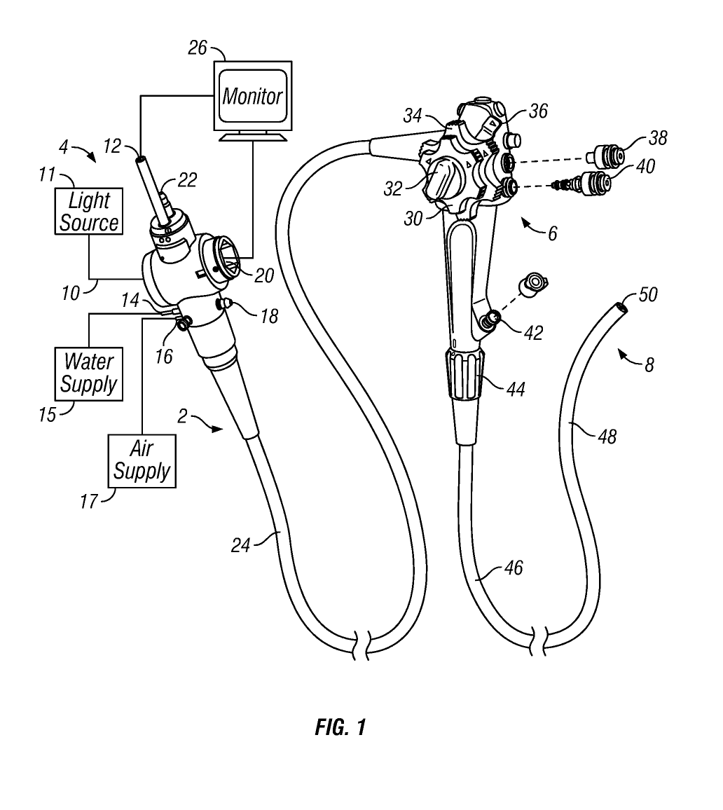

FIG. 1 is a schematic diagram of an exemplary endoscope. The terms “endoscope” or “scope” are used broadly in this application and include any tool insertable into a body having a channel through which tools and other devices can be placed and used, whether inserted through a natural body orifice or through an artificially created opening, such as through an incision or other procedure (generally termed “body passage” herein), and thus includes other medical scopes modified to be able to convey tools and related instruments or for viewing of internal body tissues. Thus, for present purposes, references to endoscopes, and more generally scopes also include enteroscopes, esophagoscopes, colonoscopies, laparoscopes, pediatric scopes, choledochoscopes, pancreato scopes, esophagogastroduodeno (EGD) scope, and so forth.

The endoscope 2 of varying sizes can be inserted through portions of a patient's body to illuminate and remotely view internal portions of the body. The endoscope can be a single channel endoscope or a multi-channel endoscope having two or more channels. The endoscope 2 generally includes a connector portion 4 flexibly coupled to a control portion 6 which can control a distal portion 8 that is inserted into the body opening. The connector portion 4 generally includes various connectors for connecting sources of light, water, air, suction, and so forth to the endoscope 2. For example, the connector portion 4 can include a light source connector 10 that could be coupled to a light source 11. The light source connector 10 can be coupled to a remote lamp as a light source that provides elimination through the light guide 12. The connector portion 4 can further include a water supply connector 14 that can be coupled to a water supply 15 and used to flow water through the scope and out the distal end to irrigate the scope lens or tissue surfaces to which the scope is adjacent. An air supply connector 16 that can be coupled to an air supply 17 and can provide air into the endoscope for insufflations and other purposes. A suction connector 18 can withdraw fluids, such as water and other fluids from the scope. A video processor connector 20 can be used to connect a monitor 26 or other output for viewing or other purposes. An air pipe 22 can be used to supply or control air into the scope and then to the body. The connector portion 4 also includes a cord 24 that is flexibly connected to the control portion 6. The cord 24 directs various conduits coupled to the connectors described above to the control portion and thence to a distal tip 50 which is inserted into the body opening.

The control portion 6 is used to control the distal end of the scope by various angulation controls. For example, the control portion 6 can include a right/left angulation knob 30. The angulation knob 30 is jointly coupled to a pair of control angulation wires that extend towards the distal tip 50. The physician can rotate the angulation knob 30 counter-clockwise or clockwise to provide tension to an angulation wire which can remotely move the tip to the right or left. A right/left angulation lock 32 can be used to lock the right/left angulation at a fixed position. Similarly, an up/down angulation knob 34 moves the distal tip 50 in an up and down direction, generally orthogonal to the right/left angulation knob. In a similar fashion, the up/down angulation knob 34 can be rotated forward and backward to move the distal tip 50 to various angles. Similarly, an up/down angulation lock 36 can be used to lock the knob and relative angle into position. Rotating the up and down and right and left knobs together can produce a combined tip movement and allows the physician to sweep the tip of the scope in a variety of directions.

The control portion 6 can also include a suction valve 38 and a vent hole 40. The particular position of the valve 38 and vent hole 40 are such that the physician can selectively close the opening with their fingertips to control the suction and venting of the endoscope. The control portion further includes a channel 42. The channel 42 allows instruments, clamps, and other devices to be inserted through the endoscope and protrude from the distal tip 50 to the tissue area in question.

The endoscope further can include a stiffness control 44. Generally, the stiffness control can be rotated clockwise or counter-clockwise to adjust the relative stiffness of the distal portion 8 within a given range. An insertion tube 46 is coupled to the control portion 6 and further includes the various conduits for the water, air, light, and so forth. Multiple conduits can be used for multiple lights, image capturing and other functions. A distal tip 50 is the scope portion from which the various conduits for air, water, and so forth as well as the channel 42 can be disposed. A bending section 48 is particularly adapted to bend using the control wires from the angulation knobs 30, 34. The bending allows greater mobility in navigating through the curves of the body passages.

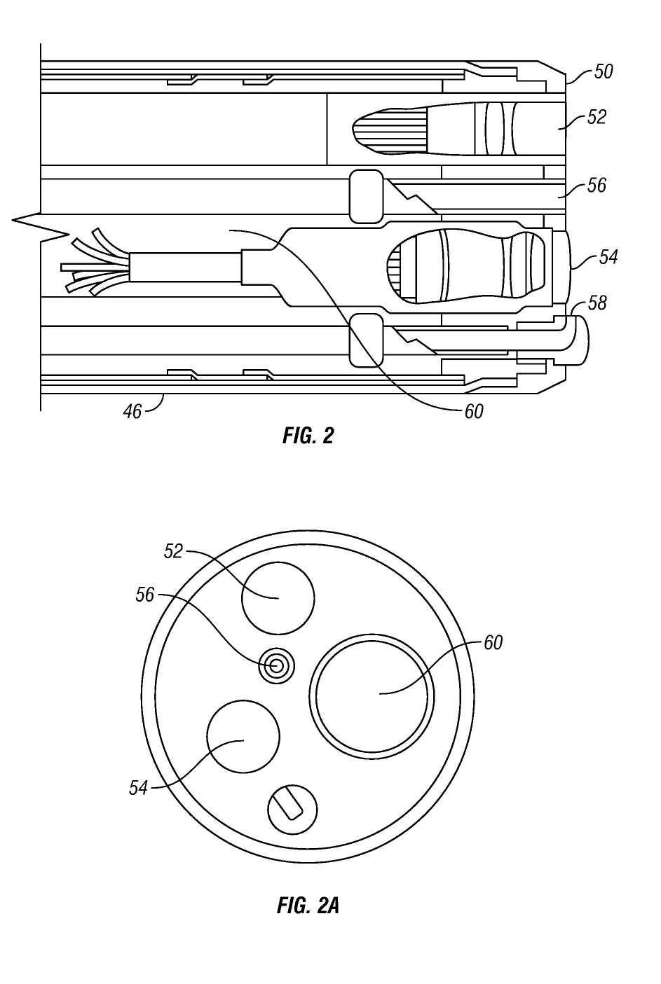

FIG. 2 is a schematic diagram of an exemplary-longitudinal cross-section of a distal end of the endoscope of FIG. 1. FIG. 2A is a schematic diagram of a distal end of the endoscope of FIG. 1. The figures will be described in conjunction with each other. Similar elements as described above are similarly numbered herein. The distal tip 50 generally could include one or more openings, and light and image lens. For example, the distal tip can include a water jet 56 coupled to the water supply connector 14 described above. The distal tip can further include an air and/or water nozzle 58. The light source connector 10 can provide light from the light source 11 described above to a light lens 52 through one or more optic fibers to spread the light at the tip. The light can guide the physician in viewing the tissues through which the endoscope is directed. To view the illuminated tissues provided by the light lens 52, an objective lens 54 can be provided in the endoscope. The objective lens 54 can be coupled to the monitor 26, the light guide 12, and other output elements. The objective lens focuses a miniature image of the tissue on the surface of the solid state CCD image sensor. The image sensor sends back images to a video processor to a collection of very fine electrical wires. Light illuminates the interior of the body through fiber-optic illumination fibers and the light can be easily disbursed across the endoscope's field of view by a light guide lens system. Some instruments have two fiber optic bundles and two light guide lens to provide illumination on both sides of the various tools, such as biopsy forceps, snares and so forth. The insertion tube also contains small tubes that carry air and water through the instrument. These tubes generally merge into single tubes a few inches from the distal tip. The combined ear water tube connects to an air water nozzle on the tip of the instrument. The physician can feed the water across the objective lens to clean it and air can be fed from the nozzle to insufflate the area of the body through which the scope is positioned. The endoscope further includes a channel 60 for inserting tools and other devices for tissue re-suction, biopsy, sutures, clips, and other instruments as may be appropriate for the particular procedure. The devices may have further elements. For example, the clips can be configured with multi-angle stainless steel ribbons. While the objective lens and light lens are shown on the longitudinal end of the distal tip, it is to be understood that the elements can be disposed to the side of the end at the distal tip so that a sideways view can be obtained.

As described above, the typical challenge with an endoscope is providing a small enough outer diameter to travel through the body passages, and yet still allow a large enough channel through which instruments can be routed. On a typical mother-daughter arrangement, the channel 60 is too small for a daughter scope that can, in turn, have its own channel sufficient large for routing various tools and other instruments.

The present invention solves this challenge by creating a scope carrier that can function as a partial endoscope in that it can be directed through a body passage and contains a channel for a scope, tool, or other device to be inserted therethrough. However, the scope carrier can have a relatively much larger channel than for example a “mother” scope would have in combination with a “daughter” scope. The larger carrier channel allows use of a larger scope with a larger scope channel to be inserted into the carrier channel. The larger scope channel in turn allows use of instruments and procedures that heretofore have been unavailable in such combinations. Such a scope channel can also be used to retrieve material from the inserted scope, including biopsies, resected tissue and so forth.

The system can be used for examining small bowels or examining a colon that cannot be reached with conventional scopes. Further, the system can be used to examine the bile ducts or pancreas ducts, right colon or various convoluted angles of the stomach or colon that heretofore are complicated due to multiple bending functions. By passing the scope directly into the bile duct or pancreas duct, a direct view of the interior of the duct lining can be made, so that suspected duct abnormalities can be examined in greater detail. The invention can be used for other medical procedures, including exploratory procedures in the gastrointestinal tract, thoracic cavity, abdominal cavity, genital and urinary tract, cranial cavity, and other body areas. With the present disclosure and system therein, biopsies, and other procedures can be made. In general, the scope is coupled to the scope carrier in a first mode so that during insertion of the carrier, the light lens and objective lens of the scope are used to guide the scope carrier for the physician. When the carrier is properly positioned, the scope can be released from the carrier in a second mode and continue further through the body passages to the intended tissue or structure.

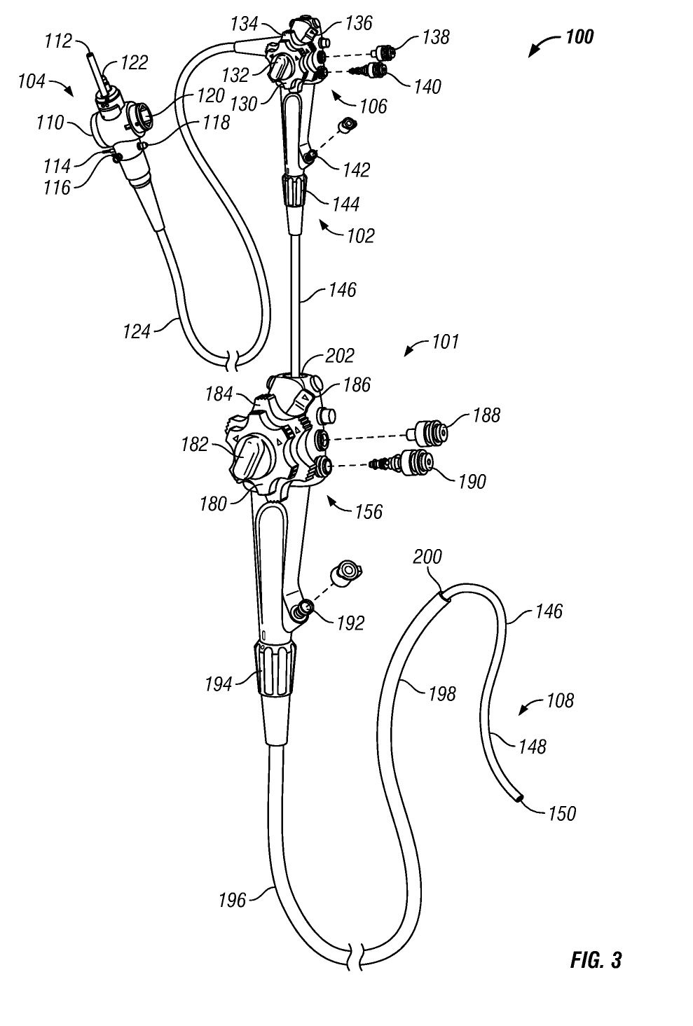

FIG. 3 is a schematic perspective diagram of an exemplary endoscopic system of the present invention with a scope carrier and a scope inserted therein. The endoscope 102 such as described in FIGS. 1-2A can be inserted through a scope carrier 101. More particularly, the endoscope 102 can be of various typical sizes of endoscopes sufficiently large enough to have a channel that can deliver a tool or other instrument therethrough. This differs markedly from prior mother/daughter scopes assemblies, because the scope carrier does not rely on its own optics, light, water and air conduits to perform its function. Instead, the scope carrier relies upon the scope inserted therethrough to guide the physician in orienting the scope carrier to a proper position. In some embodiments, the scope can be released from the scope carrier and travel further into the body cavity using its own light and optic elements.

In general, the scope 102 includes elements of the scope 2 described in FIG. 1. For example, the scope 102 can include a connector portion 104 coupled to a control portion 106, and the control portion 106 coupled to a distal portion 108 having a distal tip 150. The connector portion can include a light guide 112, water supply connector 114, an air supply connector 116, a suction connector 118, a video processor connector 120, and an air pipe 122 that can provide the connections of the scope to the external support elements such as water supply, air supply, suction supply, video processor and so forth.

The control portion 106 is coupled to the connector portion 104. The control portion 106 similarly includes elements as described for the scope 2 of FIG. 1. For example, the control portion 106 can include a right/left angulation knob 130 and corresponding lock 132, as well as an up/down angulation knob 134 and corresponding lock 136. The scope can include a suction valve 138 and a vent hole 140 that can help control the air/water delivery to the distal tip 150. The scope 102 generally includes a channel 142. The channel in the scope 102 is generally sized sufficiently large enough to allow instruments and other tools and devices to be delivered down through the scope insertion tube 146 and out the distal end 150 for performing various medical procedures, such as tissue re-suction, biopsy, and other features that heretofore have not been readily available to the physician. The scope 102 can also include a stiffness control 144 for the stiffness of the insertion tube 146, and/or bending section 148. The insertion tube 146 can be disposed in the scope carrier 101 and at some steps in the medical procedures extend through the scope carrier to continue on deeper into the body passages, such as the colon, to be inspected and any medical procedure performed thereon.

The scope carrier 101 includes a carrier channel 202 formed therein through which the endoscope 102 is disposed. The carrier channel 202 generally extends longitudinally through an upper end of the scope carrier to a distal tip of the scope carrier. The relatively large channel 202 in the scope carrier 101 allows a larger insertion tube 148 of the scope 102 to be disposed down through the carrier channel. Due to the relatively large size of the scope inserted in the scope carrier, it may be advantageous to have an axial entry point into the scope carrier through the top, as shown in FIG. 3. Alternatively, a second slim endoscope or optic fiber can be inserted through a channel 192 optionally formed in the scope carrier.

The scope carrier can include a carrier control portion 156. The carrier control portion 156 can include one or more angulation adjustments similar to the angulation adjustments on the scope 102 described above. For example, a right/left angulation knob 180 and corresponding lock 182 can be rotated clockwise or counter-clockwise to adjust the carrier angulation as it passes through various body passages. The scope carrier can also include an up/down angulation knob 184 and corresponding lock 186. In general, the channel 192 similar to the channel used in scope 102 may be unnecessary in the scope carrier 101. The scope 102 can enter the scope carrier 101 through the carrier channel 202. However, in some embodiments, an additional channel 192 can be useful to be provided in the scope carrier. Thus, the channel 192 is shown and described for such instances. Similarly, the control portion 156 will generally not need a suction valve 188 and vent hole for air and/or water 190 as has been described in respect to the scope 102. However, in some embodiments, such additional valves could be useful and are included in this description, for example, if there is an additional channel 192. One or more additional channels can be provided for multiple light guides and image capture and insertion of devices. If stiffness adjustments are appropriate for the particular scope carrier, a stiffness control 194 can also be included.

The scope carrier 101 further includes an insertion tube 196. The insertion tube 196 is larger than the scope insertion tube 146 so that the insertion tube 146 can be inserted therein, but still small enough to travel through various body passages. The relative outer diameters of the scope carrier insertion tube 196 and the scope insertion tube 146 can be closer than is customary to allow a relatively larger insertion tube 146 for a given diameter of the insertion tube 196. This closer size correspondence can occur, for example, by using the lens and light of the scope 102 for the scope carrier, and thus can provide a relatively larger carrier channel 202 for a given outer diameter of the insertion tube 196.

The suction tube 196 can include a bending section 198 that is coupled to the angulation control as described above. The insertion tube 196 terminates at a distal tip 200. The distal tip 200 can include a longitudinal opening, an obtuse angle opening, or a side channel. The particular embodiments shown in FIG. 3 include a longitudinal opening. However, it is to be understood that one or more additional openings at different angles to the longitudinal opening are contemplated.

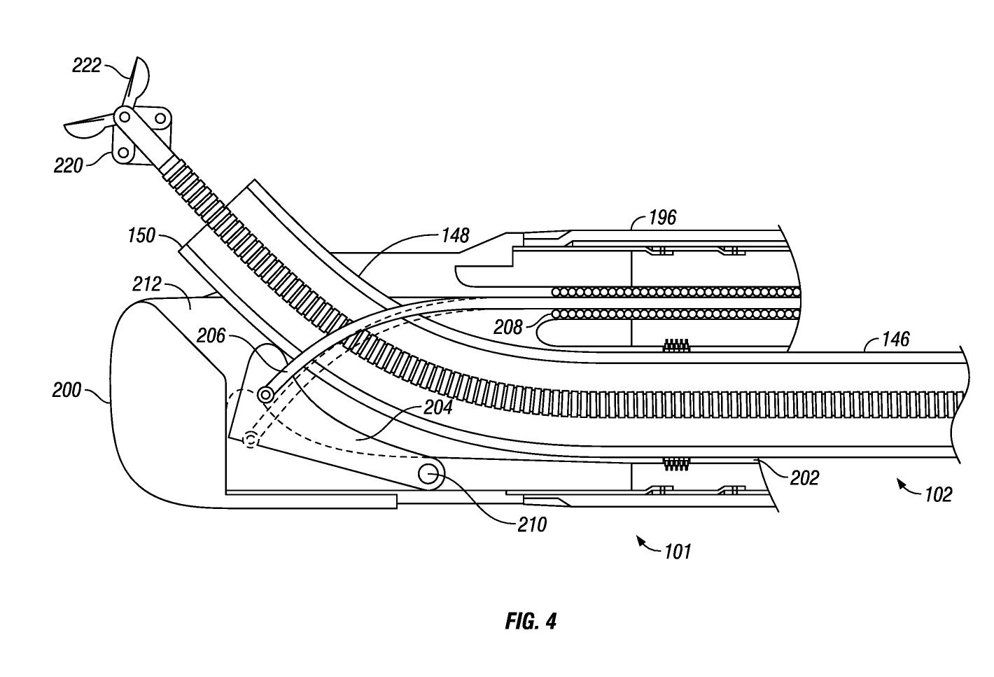

FIG. 4 is a schematic longitudinal cross-sectional diagram of an exemplary embodiment of the scope carrier having a distal tip with an elevator. The scope carrier 101 can include an elevator to direct the exit of the distal tip of the scope inserted therethrough. More particularly, the insertion tube 196 can include an opening 212 of the carrier channel 202 through which the distal tip 150 of the scope 102 can exit the scope carrier 101. As seen in FIG. 4, the relatively small difference in outer diameters between the scope carrier insertion tube 196 and scope insertion tube 146 can allow for a relatively large scope 102 with a correspondingly large channel 142 to be inserted through the carrier channel 202. The large channel therein can allow for a sufficiently large scope instrument 220 to be disposed therethrough to perform various medical procedures that heretofore have been difficult to perform due to limited size of the channel in the scope. As merely one example, the scope instrument 220 can include one or more resector blades 222 that can be used to perform biopsies or remove various tissues as part of a given medical procedure. For example and without limitation, the larger channel of the scope can be used to remove large polyps deep into the interior body passages. The large polyps can be excised and pulled into the carrier channel along with the scope 102. If necessary, the scope 102 can be removed from the carrier 101 while the carrier is left in position to remove the excised material. The scope can then be reinserted into the already positioned scope carrier for further inspection and/or removal. Other tools, such as snares, clips, and other instruments can be disposed through the channel 142 of the insertion tube 146.

As a further example, the scope carrier and scope can be inserted into the duodenum and maneuvered to the anpulla. The scope can be unlocked from the relative position to the scope carrier 101 and extended from the scope carrier into the bile duct of the patient. The larger channel of the scope allows therapy to be accomplished with various devices or instruments insertable therethrough.

The distal tip 200 of the scope carrier 101 can include one or more elevators 204 for assisting and directing the exit of the insertion tube 146 from the distal tip 200. The elevator 204 generally includes an elevator control wire 206 that is guided down the insertion tube 196 of the scope carrier 101 through an elevator wire guide 208. The elevator can pivot about an elevator pivot joint 210, so that as the elevator control wire 206 is moved longitudinally through the elevator wire guide 208, the elevator 204 is pulled and pushed longitudinally which can raise and lower the elevator as it pivots about the joint 210. Depending upon the amount of raising and lowering, the exit angle of the distal tip 150 can change.

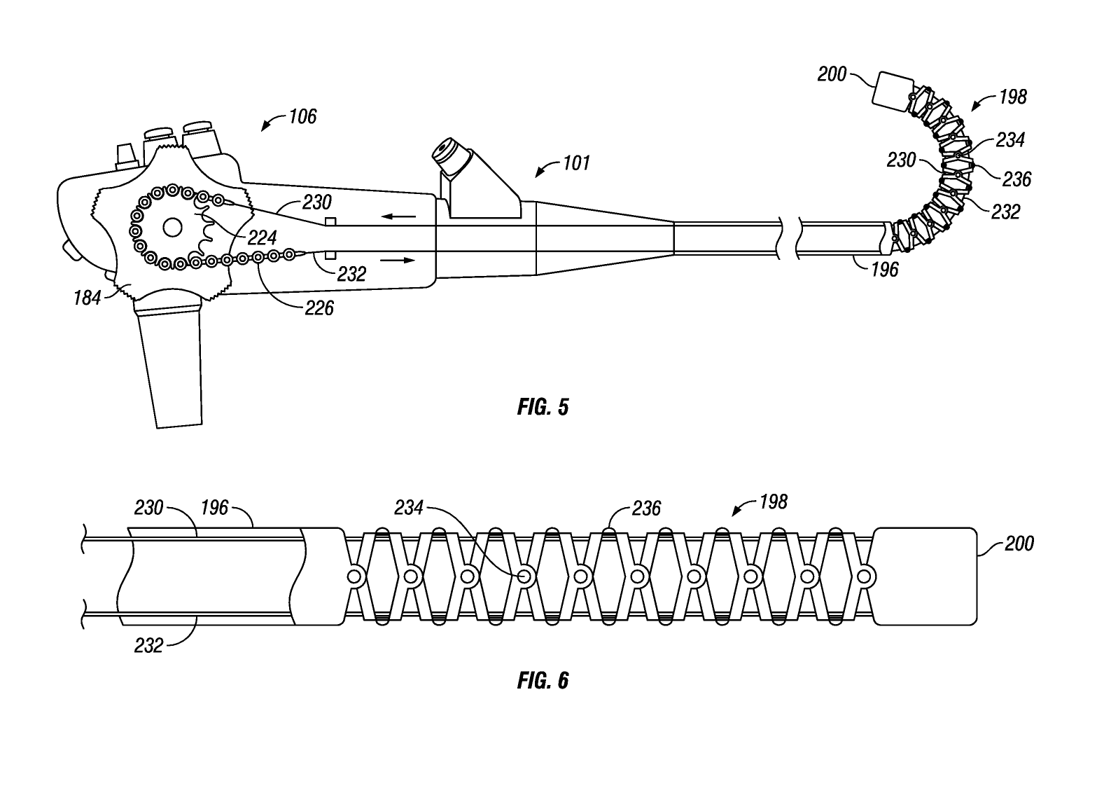

FIG. 5 is a schematic longitudinal, cross-sectional diagram of the exemplary scope carrier having angulation capabilities. FIG. 6 is a schematic longitudinal, cross-sectional diagram of an angulation structure of the scope carrier of FIG. 5. These figures will be described in conjunction with each other. The scope carrier 101 can include angulation controls and structure as has been described above for the scope 102. For example, and without limitation, the scope carrier 101 can include a control section 106 that internally can include one or more sprockets 224 with a chain 226 disposed therearound. The chains 226 can be attached on each end to a pair of angulation wires 230, 232. For example, one of the angulation wires can be an up angulation wire 230 and the other angulation wire can be a down angulation wire 232. The rotation of the sprocket 224 can be controlled by an externally operated up and down angulation knob 184, described above. The angulation wires are coupled to a bending section 198 of the insertion tube 196. The bending section 198 can include a plurality of up/down angulation pivot links 234. When the sprocket 24 is rotated in a counter-clockwise direction according to the view in FIG. 5, the up angulation wire is tightened, causing the links 234 to come in closer proximity to each other and thus bend the distal tip 200 in an up direction according to orientation in FIG. 5. At the same time, the down angulation wire 232 is loosened which allows the link 234 to expand on the lower portion of the bending section 198 to allow the bending of the distal end 200 to be more fully realized. Similarly, when the sprocket 224 is rotated in a clockwise direction, the down angulation wire 232 is tightened, causing the link 234 to be compressed on the lower portion while the up angulation wire 230 is loosened and allows the link 234 to be opened more fully on the upper portion of the bending section 198. The relative movement of the angulation wire causes the distal tip 200 to be rotated in a downward direction. Similarly, the bending section 198 can include one or more left/right angulation pivot links 236 for movement of the bending section in a left/right direction, that is, at an angle to the view shown in FIG. 5. The left/right angulation pivot links can be coupled to similar angulation wires, knobs, and so forth.

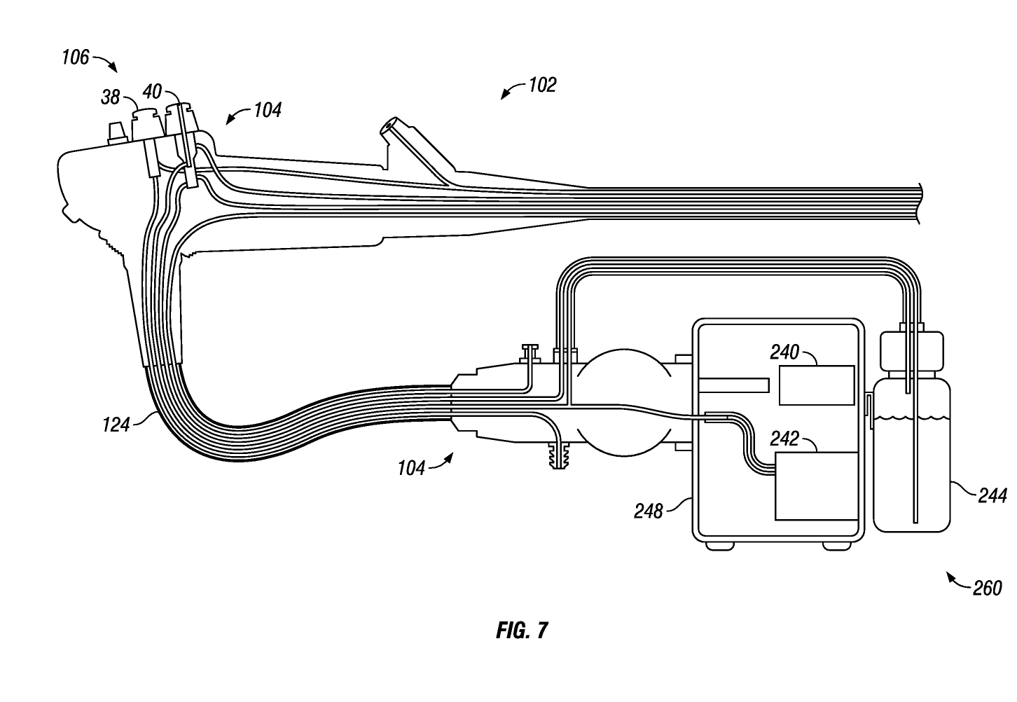

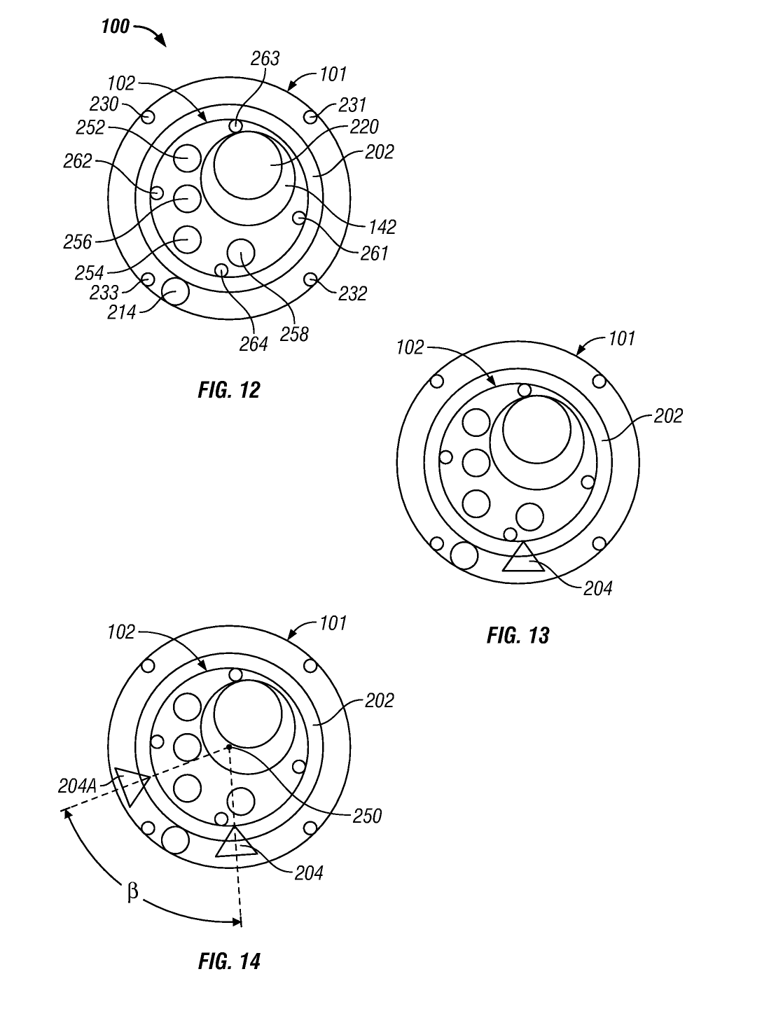

FIG. 7 is a schematic diagram of supply sources for the scope. By way of illustration and without limitation, various supply sources 260 can be coupled to the scope 102 in the connector portion 104. For example, a lamp 240 can provide light through the light guide 252, shown in FIG. 12. An air pump 242 can provide air to the air supply connector 116, shown in FIG. 3.

Further, a water supply 244 can be provided to the connector portion 104. The connector portion 104 of the scope 102 can provide manifolding of the various supply sources 260 to be coupled through the cord 124 to the control portion 106 of the scope. A housing 248 can surround and offer protection or contaminant resistance to the various supply sources described herein.

The air pump 242 can provide air under mild pressure to a pipe protruding from the endoscope's light source. The air is carried by an air channel to the air/water valve 40 on the control portion 106. If the valve is not covered, the air can simply exit from a hole in the top of the valve. This hole allows air pump to pump fully when air is not needed. If the physician wants to insufflate the patient, the physician can cover the vent hole with a fingertip to close the vent and force air down the air channel. A suction is also controlled by a suction valve 38 on the endoscope's control portion. A suction supply (not shown) can be connected to the control portion. When a physician depresses the suction valve 38, suction can be applied to the channel within the insertion tube. Fluid or air at the distal tip can be drawn into the suction collection system.

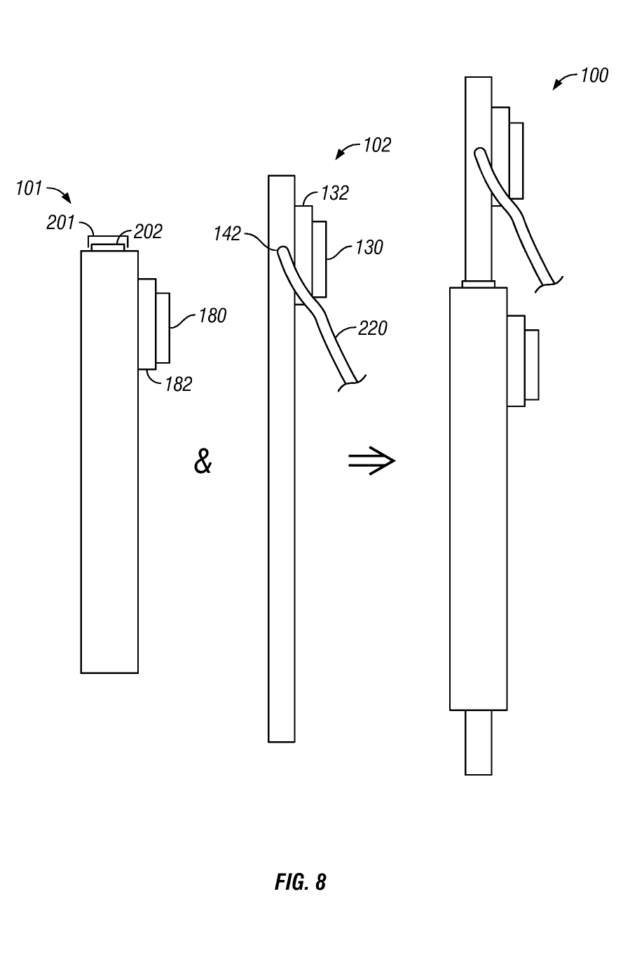

FIG. 8 is a schematic diagram of the exemplary scope carrier and scope. The next series of figures provide conceptual schematics of various aspects of the invention. In general, the assembly 100 includes a scope carrier 100 and a scope 102 slidably engaged with the scope carrier to form a system 100. The scope carrier can include an angulation control such as a right/left angulation knob 180 with an associated lock 182. It is to be understood that the angulation control can also include the up/down angulation knob 184 and associated lock 186 described above. The scope 102 can also include angulation controls, such as right/left angulation knob 130 and associated lock 132. The scope 102 can further include the channel 142 through which various tools and other instruments 220 can be inserted. A cap 201 can be coupled to the scope carrier to close, or at least partially close, the scope carrier channel 202.

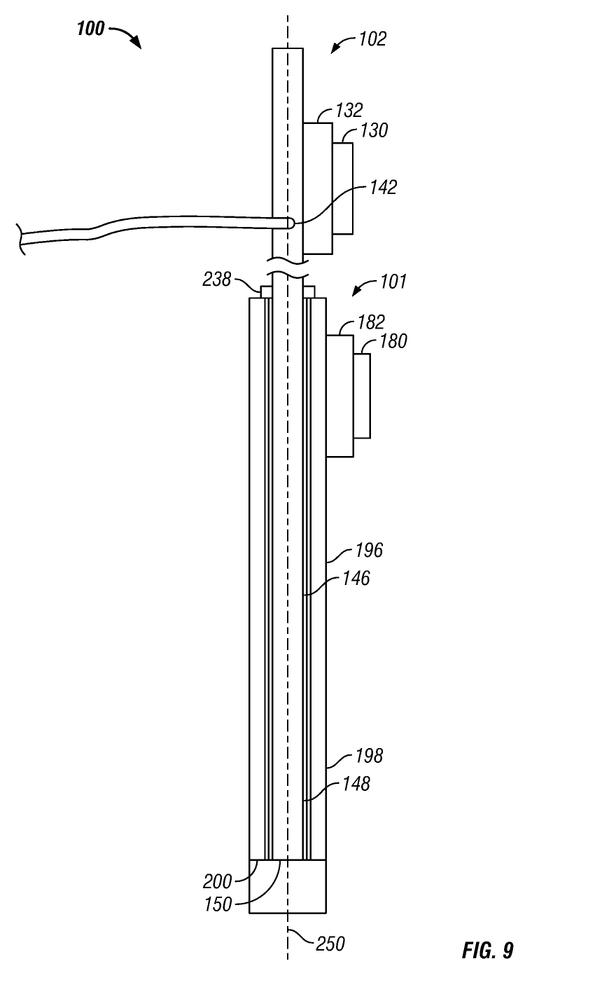

FIG. 9 is a schematic longitudinal, cross-sectional diagram of an exemplary endoscopic system. The system 100 can include the scope carrier 101 and the scope 102. In at least one embodiment, the scope carrier and scope can be coupled together in longitudinal position by a lock 238. For example and without limitation, the lock 238 can be rotatable about the scope 102 that restricts the downward movement of the scope. Thus, the distal end 150 of the scope 102 can be held in proximity to the distal end 200 of the scope carrier 101. Upon inserting the system 100 into a body passage, the light and lens described herein for the scope 102 can provide guidance to the physician as the scope carrier 200 is inserted in the body passage(s). Upon reaching a certain position, the lock 238 can be released, so that the scope 102 can move longitudinally along the longitudinal axis 250 of the scope carrier 101 and be inserted further into the body passage. It is to be expressly understood that the lock 238 is only an exemplary method of coupling and other methods are contemplated including a slidable sleeve over the scope or manual holding of the scope in a relative fixed position to the scope carrier during such insertion.

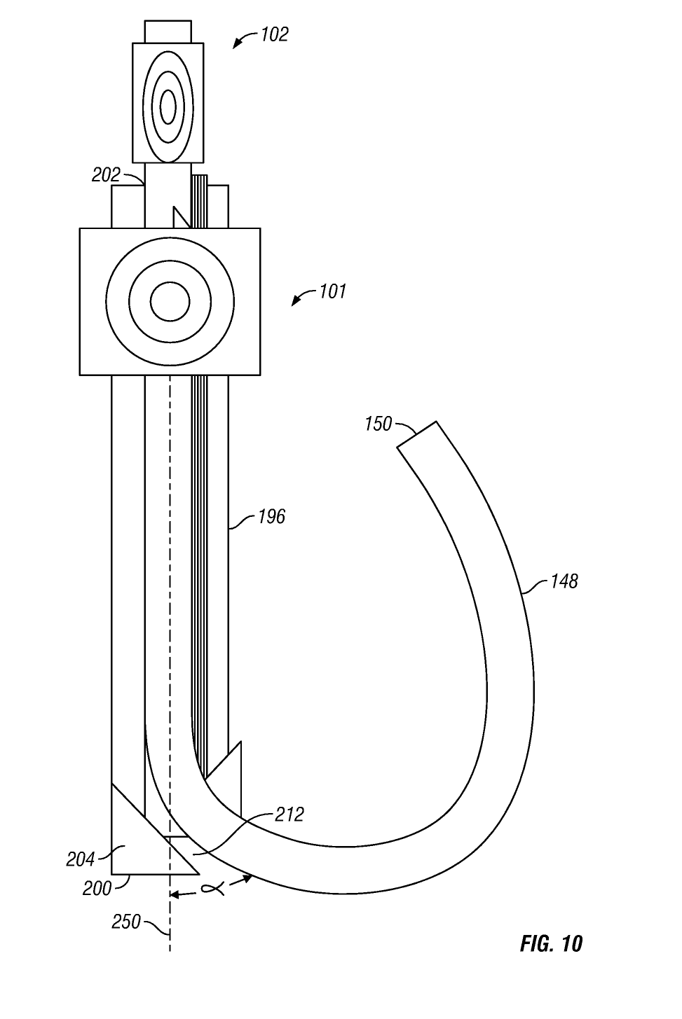

FIG. 10 is a schematic longitudinal, cross-sectional diagram of the endoscopic system with an elevator and angulation controls. The scope 102 can be inserted through the carrier channel 202 toward the distal end 200 of the scope carrier 101. An elevator 204 can direct the distal end 150 of the scope 102 through the scope carrier opening 212 at an angle “a” to the longitudinal axis 250 of the insertion tube 196 of the scope carrier 101. The elevator 204 can be activated to different angles to help guide the scope as it exits the opening 212. Further, the angulation controls of the scope 102 and/or angulation controls of the scope carrier 101 can be used to further help adjust the movement of the distal tip 150 exiting from the scope carrier 101, and continuing forward through the various body passages.

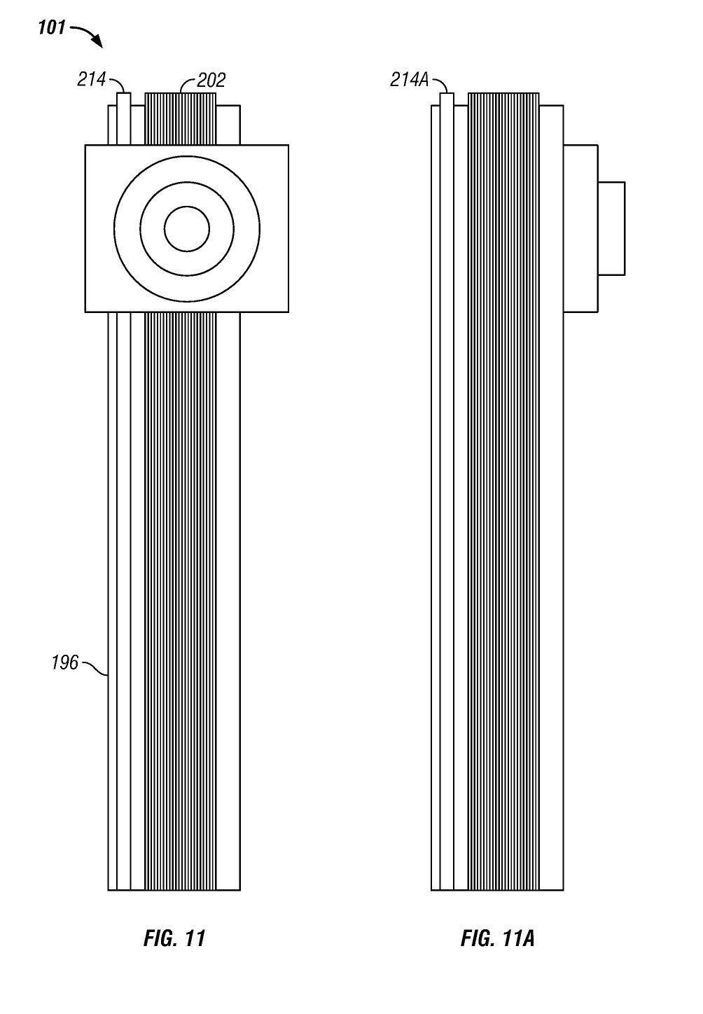

FIG. 11 is a schematic longitudinal, cross-sectional diagram of the scope carrier having a light-image capture element. FIG. 11A is a schematic longitudinal cross-sectional diagram of the scope carrier showing an additional light-image capture element from FIG. 11. The figures will be described in conjunction with each other. The scope carrier 101 can include one or more carrier light guides/objective lenses 214. The light guide/objective lens 214 can include optic fibers to carry light downward through the scope carrier and can further include a portion that is an objective lens to carry images from the generated light back up the scope carrier to an image processor, monitor, or other elements. The inclusion of such a light guide/objective lens can reduce the available cross-sectional square area in a given insertion tube 196 through which a carrier channel 202 can be formed. However, in some embodiments, such options may be desirable and overcome the need for a greater cross-sectional area of the carrier channel 202. One or more additional light guides/objective lenses 214A can also be included at various angles around the periphery of the scope carrier 101.

The scope carrier can vary in length and diameter. For example and without limitation, the scope carrier can vary from 5 cm to 220 cm, and have a diameter from 8 mm to 16 mm. In at least one embodiment, it is believed that a 12 mm scope carrier 101 can include a 10 mm carrier channel 202 to allow a scope to be inserted therethrough, representing about 80% of the cross-sectional area of the scope carrier. As another example, the outer diameter of the scope carrier can be 11.6 mm to 12.8 mm. With the present invention, the channel 202 can be, for example, occupying at least 50% and generally could be between 60% to 90% of the outer diameter of the carrier, in some embodiments about 75% to 85%, and in some embodiments about 80%. The length of the scope carrier could, for example, be 70 cm; however, shorter and longer lengths are available. Further, it is believed that the size of the scope insertable through the 10 mm carrier channel could be up to about 9 mm. A 9 mm scope generally has up to about a 3.2 mm channel, large enough to allow many endoscopic instruments to be inserted therethrough. The length of the scope can vary from 5 cm to 250 cm, and the outer diameter of the scope can generally vary from 3 mm to 9 mm. As a further example, the scope 101 can be a EGD scope with a 3.2 mm channel. Other sizes of channels, scopes, and carriers can be used and the above examples are given to illustrate the beneficial effects of the increase in available cross sectional square area by use of the scope carrier 102.

Such a large percentage of the available cross sectional area for the channel 202 sharply contrasts with existing systems, such as a mother-daughter scope assembly, where a 12 mm mother scope might have a capacity of a 4.8 mm channel through which the outer diameter of the daughter scope must be inserted . . . . These representative diameters calculate to a channel size of about 40% of the cross sectional area of the mother scope outer diameter. A standard 9 mm scope generally has up to about a 3.2 mm channel for about 36% of the cross sectional area. A daughter scope capable of insertion into the mother scope could be about 3 mm to 4 mm outer diameter and would itself generally have a channel size of about 1 mm, about 25% to 33% of the cross sectional area. This daughter channel size is smaller than several types of currently available endoscopic instruments can accommodate.

FIG. 12 is a schematic cross-sectional diagram of an exemplary system having the scope carrier and the scope. The system 100 can include the scope carrier 101 having a carrier channel 202 through which a scope 102 is slidably disposed therein. The scope carrier can include one or more guide wires, such as up angulation wire 230, down angulation wire 232, left angulation wire 231, and right angulation wire 233. Further, the scope carrier 101 can include a carrier light guide/objective lens that can include a light source, optic fibers, and an objective lens to return the images generated through the light source at the distal end of the carrier. The scope 102, inserted in the carrier channel 202, can include also one or more angulation wires 261, 262, 263, 264. The scope 102 includes its own scope channel 142 through which one or more instruments 220 can be inserted. The instruments can include various devices for resecting tissue, biopsies, clamps, clips, sutures, and so forth. The scope 102 can further include a light guide 252, objective lens 254, water jet 256, and/or water nozzle 258, similar to the elements described above in reference to FIGS. 2 and 2A. The carrier channel 202 is a relatively large channel compared to the outer diameter of the carrier 101. The relatively larger carrier channel 202 allows a larger scope 102 with a correspondingly larger scope channel 142 to be inserted therethrough which in turn allows a larger instrument 220 to be inserted through the scope channel 142. The relative diameters allow use of tools heretofore not believed to have been available due to the relatively limited, small scopes that have previously been used with their relatively small channels, when such scopes are inserted through other scopes, such as a mother scope.

FIG. 13 is a schematic cross-sectional diagram of the exemplary system with an elevator. The cross-section of the carrier 101 and scope 102 illustrates a schematic form of an elevator 204 that can be at least partially engaged with the scope 102 and change the angle of the scope 102 as it exits the scope carrier 101.

FIG. 14 is a schematic cross-sectional diagram of the exemplary system with a plurality of elevators. This figure is similar to FIG. 13 but includes a plurality of elevators disposed at some angle “β” relative to each other. In at least one embodiment, the angle can be 90 degrees, although greater and smaller angles can be used. In general, the use of a plurality of elevators 204, 204A can allow for increased control of the angle to which the scope 102 exits the scope carrier 101. For example, the scope can be controlled to exit the carrier 101 at a variety of angles in an X-Y plane that is transverse to the longitudinal axis 250 through the scope carrier channel 202.

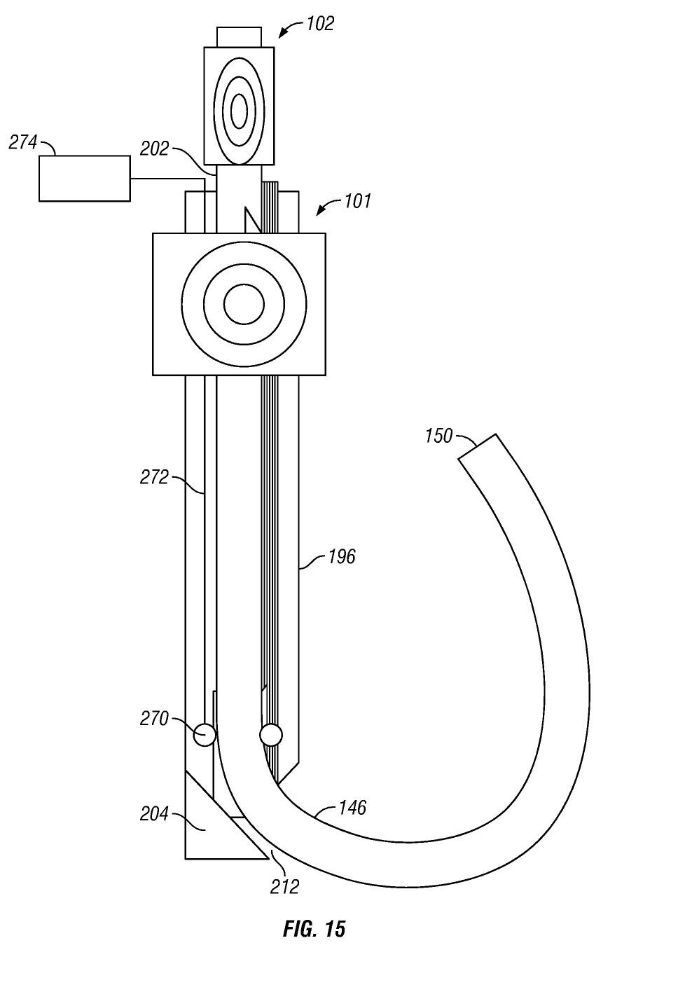

FIG. 15 is a schematic cross-sectional view of the endoscopic system with rollers. In some embodiments, the scope carrier and/or scope can include one or more roller elements 270. A roller element 270 can include ball bearings, roller bearings, sleeve bearings, sleeves, and other elements on which the scope 102 can roll through at least a portion of the scope carrier 101. For example and without limitation, a stress zone can be created between the scope carrier 101 and the scope 102 at the scope carrier opening 212, where the scope insertion tube 146 can exit the scope carrier. A roller element 270 can assist in reducing frictional forces and allow better control and movement of the scope through the scope carrier. The less friction can be especially useful if the scope is exiting the scope carrier at a certain angle, such as in conjunction with the use of an elevator 204. Further, in some embodiments the roller element 270 can be powered. A control conduit 272 can provide power and/or control to the roller element 270. A controller 274 can be used to actuate and otherwise control the activation and deactivation of the roller element 270. Without limitation, the roller element 270 could include an air motor, and a control conduit 272 could include an air conduit to provide air to the air motor. The control conduit 272 could also include electrical and other power sources. The location of the roller element 270 can vary and be toward the end of the carrier channel 202 as shown, the beginning of the carrier channel, or in some intermediate location. For example, it may be useful to have roller elements distributed along the carrier channel 202 at various locations. The distribution can help reduce friction caused by the sliding movement of the scope 102 through the carrier channel 202, particularly through the various bending that occurs as the scope and carrier move through the body passages.

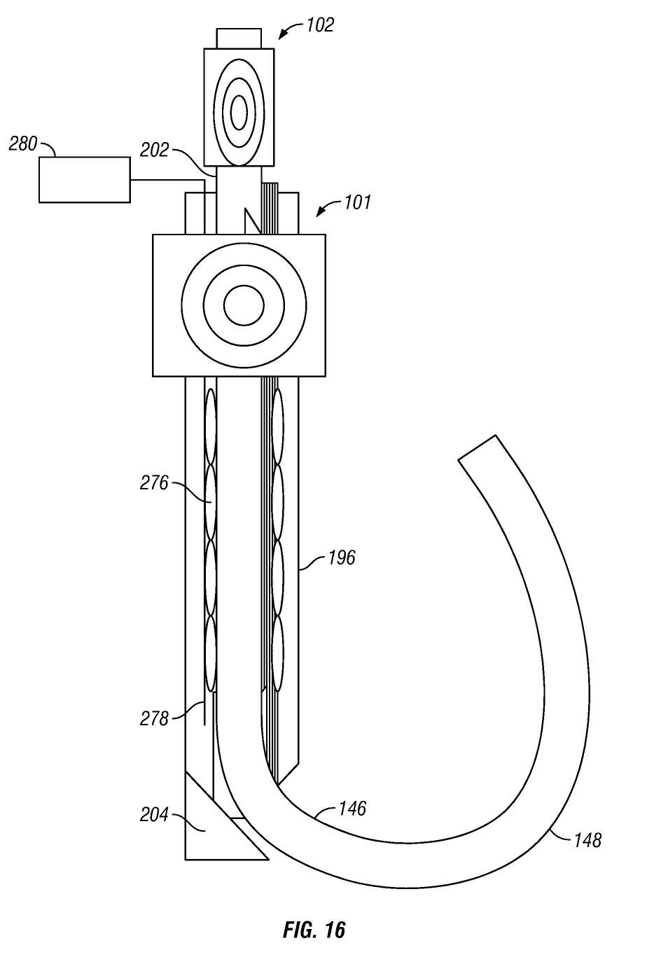

FIG. 16 is a schematic cross-sectional view of the system with inflatable portions. A further embodiment of the scope carrier can include compartmentalized inflatable portions of the carrier channel 202. The inflatable portions can be integral to the carrier channel or can be removably disposed therein. The inflatable portions can be selectively inflated and deflated by providing a conduit 278 to the one or more inflatable portions. The inflatable medium, such as air, can be controlled by a controller 280. The controller 280 can include a microprocessor or other processing element to determine the selective activation and deactivation. The selective activation and deactivation and resulting inflation and deflation can push the scope 102 backward and forward relative to the scope carrier 101. Such movement may assist the physician in deploying the scope 102 from the scope carrier 101.

The invention has been described in the context of various embodiments and not every embodiment of the invention has been described. Apparent modifications and alterations to the described embodiments are available to those of ordinary skill in the art. The disclosed and undisclosed embodiments are not intended to limit or restrict the scope or applicability of the invention conceived of by the Applicant, but rather, in conformity with the patent laws, Applicant intends to protect all such modifications and improvements to the full extent that such falls within the scope or range of equivalents of the following claims.

The various methods and embodiments of the invention can be included in combination with each other to produce variations of the disclosed methods and embodiments, as would be understood by those with ordinary skill in the art, given the understanding provided herein. Also, various aspects of the embodiments could be used in conjunction with each other to accomplish the understood goals of the invention. Also, the directions such as “top,” “bottom,” “left,” “right,” “upper,” “lower,” and other directions and orientations are described herein for clarity in reference to the figures and are not to be limiting of the actual device or system or use of the device or system. The term “coupled,” “coupling,” “coupler,” and like terms are used broadly herein and can include any method or device for securing, binding, bonding, fastening, attaching, joining, inserting therein, forming thereon or therein, communicating, or otherwise associating, for example, mechanically, magnetically, electrically, chemically, directly or indirectly with intermediate elements, one or more pieces of members together and can further include without limitation integrally forming one functional member with another in a unity fashion. The coupling can occur in any direction, including rotationally. Unless the context requires otherwise, the word “comprise” or variations such as “comprises” or “comprising”, should be understood to imply the inclusion of at least the stated element or step, or group of elements or steps, or equivalents thereof, and not the exclusion of a greater numerical quantity or any other element or step, or group of elements or steps, or equivalents thereof. The device or system may be used in a number of directions and orientations. Further, the order of steps can occur in a variety of sequences unless otherwise specifically limited. The various steps described herein can be combined with other steps, interlineated with the stated steps, and/or split into multiple steps. Additionally, the headings herein are for the convenience of the reader and are not intended to limit the scope of the invention.

Further, any references mentioned in the application for this patent as well as all references listed in the information disclosure originally filed with the application are hereby incorporated by reference in their entirety, to the extent such may be deemed essential to support the enabling of the invention. However, to the extent statements might be considered inconsistent with the patenting of the invention, such statements are expressly not meant to be considered as made by the Applicant(s).

1. A medical endoscope system, comprising:
a scope carrier comprising a cylindrical tube having a carrier channel formed therein and adapted to allow a scope to be inserted therethrough; and
a scope comprising an objective lens and a scope channel, the scope being adapted to be slidably disposed within the carrier channel.

2. The system of claim 1, wherein the carrier channel is at least 50% of the cross sectional square area of an outer diameter of the scope carrier.

3. The system of claim 1, wherein the scope carrier is adapted to use the objective lens of the scope to guide the scope carrier into a body passage.

4. The system of claim 3, wherein the scope carrier excludes an objective lens formed within the scope carrier.

5. The system of claim 1, wherein the scope carrier is adapted to be coupled to the scope in a fixed longitudinal position during a first mode while the scope carrier is inserted into a body passage and decoupled from the scope in a second mode after the scope carrier is inserted into the body passage to allow the scope to be slidably disposed within the scope carrier.

6. The system of claim 1, wherein the carrier channel extends longitudinally through an upper end of the scope carrier to a distal tip of the scope carrier.

7. The system of claim 1, wherein the scope carrier comprises at least one angulation control.

8. The system of claim 1, wherein the scope carrier comprises at least one elevator.

9. A medical endoscope system, comprising a scope carrier comprising a cylindrical tube having a carrier channel formed therein and adapted to allow an scope to be inserted therethrough, the carrier channel being at least 50% of the cross sectional square area of an outer diameter of the scope carrier.

10. The system of claim 9, wherein the scope carrier is adapted to be coupled to the scope in a fixed longitudinal position during a first mode while the scope carrier is inserted into a body passage and decoupled from the scope in a second mode after the scope carrier is inserted into the body passage to allow the scope to be slidably disposed within the scope carrier.

11. The system of claim 9, wherein the scope carrier excludes a light guide and objective lens formed within the scope carrier.

12. The system of claim 9, wherein the scope carrier comprises at least one angulation control.

13. The system of claim 9, wherein the scope carrier comprises at least one elevator.

14. A method of inserting a medical scope and scope carrier into a body passage, comprising:
inserting a medical scope into a scope carrier;
guiding the scope carrier into the body passage using an objective lens on the scope to guide the scope carrier;
positioning the scope carrier; and
extending the scope from the scope carrier to travel further into the body passage.

15. The method of claim 14, further comprising bending a distal portion of the scope carrier with at least one angulation wire disposed in the scope carrier while guiding the scope carrier into the body passage.

16. The method of claim 14, further comprising adjusting an angle at which the scope extends from the scope carrier by elevating an elevator disposed in the scope carrier.
